(19) United States
(12) Patent Application Publication
(10) Pub. No.: US 2026/0007342 A1
(43) Pub. Date: Jan. 8, 2026

(54) Blood Sampling System

(71) Applicant: Assaya LLC, Roswell, GA (US)

(72) Inventors: Clas SIVERTSEN, Lilburn, GA (US); Brant GIRARD, San Bruno, CA (US)

(73) Assignee: Assaya LLC, Roswell, GA (US)

(21) Appl. No.: 18/764,140

(22) Filed: Jul. 3, 2024

(51) Int. Cl. A61B 5/15; A61B 5/151

(52) U.S. Cl. CPC A61B 5/150022; A61B 5/150274; A61B 5/150305; A61B 5/150358; A61B 5/150412; A61B 5/15105

(57) ABSTRACT

The present invention relates to an innovative blood collection system including a blood collection device, or blotter, designed to efficiently and in an environmentally responsible manner collect blood samples from finger prick or other wounds, and then optionally provide a convenient vehicle for the blood sample to be sent for processing and analysis without having to transport the patient to a clinic and back.

### FIELD OF THE INVENTION

The present invention pertains to the field of medical devices, specifically to devices and methods for blood collection of whole blood, serum, or plasma for diagnostic or other purposes.

### BACKGROUND OF THE INVENTION

Blood collection is a fundamental procedure in medical diagnostics, enabling the analysis of various biomarkers to diagnose and monitor health conditions. Traditional methods of blood collection involve venipuncture by a phlebotomist. Some solutions involve the use of pipettes or capillary tubes to collect blood following a finger prick. These methods, while effective, have several limitations:

Most blood collection devices, such as pipettes and capillary tubes, are made from plastic. The widespread use of plastic in medical devices contributes significantly to environmental pollution, as these items are typically single-use and non-biodegradable. Furthermore, because these materials are contaminated by biological material they must be disposed of as hazardous material.

Traditional methods can result in blood spillage, running, or dripping from the wound site, leading to potential contamination and loss of sample integrity. This not only poses a risk of infection but also makes the collection process less efficient.

The process of collecting blood using pipettes or capillary tubes can be uncomfortable and inconvenient for patients, particularly in a non-clinical setting.

The use of single-use plastic devices generates significant medical waste, contributing to environmental degradation and increased disposal costs.

The environmental impact of traditional blood collection methods is a growing concern. The medical industry is a significant contributor to plastic waste, and the single-use nature of many blood collection devices exacerbates this issue. Key environmental concerns include the production of non-biodegradable waste from traditional blood collection devices made from non-biodegradable plastics and excess resource consumption due to the production of plastic devices.

### BRIEF DESCRIPTION OF THE DRAWINGS

The accompanying drawings illustrate various embodiments of the blotter and coagulant unit devices, providing exemplary visual representations of their structure, components, and use. These drawings will help visualize embodiments of the structure, components, and use of the devices.

FIGS. 1A-1E illustrate different embodiments of the blotter showing shape and structure.

FIG. 2 illustrates one embodiment of a clotter showing shape and structure.

FIGS. 3A-3C illustrate a sequence of images demonstrating one embodiment of a visual indicator mechanism of the blotter, showing the color change as the device absorbs blood.

FIGS. 4A-4C illustrate a sequence of images demonstrating another embodiment of the visual indicator mechanism of the blotter.

FIGS. 4D-4F illustrate a sequence of images demonstrating another embodiment of the visual indicator mechanism of the blotter.

FIGS. 4G-41 illustrate a sequence of images demonstrating another embodiment of the visual indicator mechanism of the blotter.

FIGS. 5A-5C illustrates a sequence of images demonstrating use of an aluminum transport pouch, including the tear-off section and resealing strip.

FIGS. 6A-6C are illustrations of a blood sampling kit, prior to use and after use.

FIGS. 7A-7E are illustrations of embodiments of the lancet in a protective bubble.

FIGS. 8A-8D are illustrations of one embodiment of the aluminum bag including a lancet.

FIG. 9 is a flowchart of one embodiment of manufacturing the blood sampling kit.

### DETAILED DESCRIPTION

Given the limitations and environmental impact of existing blood collection methods, there is a pressing need for innovative solutions that address these challenges. The development of sustainable, efficient, and user-friendly blood collection devices can revolutionize the field of medical diagnostics. Such innovations can reduce environmental impact, improve sample integrity, and enhance user experience.

### Overview of the Components

The present system is designed to meet these needs by providing eco-friendly alternatives that improve upon the limitations of traditional methods. These devices are made from renewable and recyclable materials, integrate advanced anticoagulant and coagulant technologies, and are designed to be both effective and user-friendly. As healthcare providers and consumers become more environmentally conscious, the demand for sustainable medical devices is expected to grow. The devices align with this trend, enhancing corporate responsibility and sustainability in the medical field. These devices are designed to meet Class II medical device standards, ensuring regulatory compliance and safety, which will facilitate their adoption and market penetration.

The present system provides a method and apparatus for blood sampling and wound management, in one embodiment. The system relates to the development of an environmentally friendly and efficient blood collection device, or blotter. The blotter is designed to replace traditional blood collection methods, such as pipettes and capillary tubes, with a more sustainable and user-friendly alternative. The blood collected is intended for use in diagnostic testing, and anticoagulants embedded within the blotter substantially prevent coagulation. In one embodiment, the system may also include a coagulant unit, or clotter, which is designed to promote rapid coagulation and wound protection. In one embodiment, both devices include a chemical impregnated into the material. In one embodiment, the blotter integrates anticoagulants like Trisodium Citrate or EDTA (Ethylenediaminetetraacetic acid), and the clotter includes coagulants such as microfibrillar collagen, silica or glass particles. Since the coagulation cascade is promoted by exposing a volume of blood to a large surface area, the structure of the cellulose fibers promotes coagulation as well.

In one embodiment, blotter and clotter utilize environmentally responsible materials and advanced technologies to address the limitations of traditional blood collection methods and blood coagulation methods. The system is designed to reduce environmental impact, enhance user experience, and improve the efficiency and safety of blood collection and/or wound management.

The blotter and the clotter are constructed from materials that are both renewable and recyclable. Such materials include in one embodiment cellulose fibers and/or fluff pulp. Cellulose fibers are derived from plants, abundant, renewable, and biodegradable. Cellulose fibers provide excellent absorbency, and in one embodiment are used as the primary material for the devices. Cellulose fibers can be derived from bark, wood or leaves of plants, or from other plant-based material. Fluff pulp is often used in absorbent pads in the food packaging industry. Fluff pulp is another renewable material that offers high absorbency and is cost-effective. In one embodiment, fluff pulp is utilized for its ability to rapidly absorb and retain blood. Fluff pulp is chemical pulp made from long fiber softwoods, in one embodiment. In one embodiment, the pulp is made with the Kraft process.

Both the devices are designed to be user-friendly. The blotter can be directly placed on the area after a finger prick, eliminating the need for additional tools like pipettes or capillary tubes, or the need for precision control. Similarly, the clotter can be quickly applied to wounds to stop bleeding.

The materials used in the devices are soft and gentle on the skin, providing a comfortable experience for users. The visual indicator mechanism in the blotter ensures that users can easily determine when enough blood has been collected, reducing guesswork, enhancing convenience, and eliminating over-collection or under-collection.

The incorporation of anticoagulants such as Trisodium Citrate and/or EDTA in the blotter prevents blood from coagulating before sufficient blood is collected for testing, or within the blotter after blood collection. This ensures that the collected blood remains in a usable state for diagnostic testing, improving the accuracy and reliability of test results.

The clotter, with its integrated coagulant, provides a quick and effective solution for stopping bleeding. This is particularly beneficial in emergency situations or for individuals with conditions that affect blood clotting.

The use of renewable and recyclable materials in the devices significantly reduces their environmental impact. By replacing traditional plastic devices with the cellulose fiber based devices and aluminum packaging, the use of these devices helps decrease medical waste and promote sustainability.

In one embodiment, environmentally friendly packaging, such as sterile aluminum pouches are used, which further contributes to the reduction of plastic waste and supports eco-conscious practices in the medical field.

The simple manufacturing processes and the use of cost-effective materials make the devices affordable to produce, in one embodiment. This ensures that these devices can be made available to a wide range of healthcare providers, including those in resource-limited settings.

By minimizing the use of non-biodegradable plastics, the blotter and clotter help reduce the costs associated with medical waste management and disposal.

The blotter is ideal for use in diagnostic testing, providing an efficient and reliable method for collecting blood samples without the risk of coagulation.

The clotter is designed for rapid coagulation, making it suitable for emergency situations, surgical procedures, and for use by individuals with bleeding disorders. It can also be used to collect blood for a use that requires the blood to be clotted, in one embodiment.

The devices have wide-ranging potential applications across various medical settings. Their design and eco-friendly nature make them suitable for multiple use cases, which could significantly impact the market for blood collection and wound management devices.

The blotter is ideal for use in routine blood tests, where small volumes of blood are required for analysis. Its ability to prevent coagulation ensures that blood samples remain in optimal condition for accurate testing. The blotter can be used in point-of-care testing scenarios, such as in clinics and doctors' offices, where quick and efficient blood collection is needed. Its ease of use and reliability make it a valuable tool for healthcare providers. It also eliminates the need for a phlebotomist, since no special skill is needed to collect the correct amount of blood painlessly from a patient.

The clotter's rapid coagulation properties make it a useful tool for first responders. It can be quickly applied to wounds to stop bleeding, providing critical support in emergency situations. In trauma care settings, the clotter can be used to manage bleeding from injuries, reducing the risk of blood loss and stabilizing patients before they reach the hospital.

Surgeons can use the clotter during operations to control bleeding, ensuring a clear surgical field and reducing the risk of complications from excessive blood loss. Post-operative care can benefit from the clotter as well, by providing a means to manage bleeding from surgical wounds, promoting faster healing and reducing infection risks.

The cost-effectiveness and simple manufacturing process of the devices make them suitable for use in resource-limited settings, providing accessible healthcare solutions where traditional methods may be too expensive or impractical. Thus, these devices can be included in medical kits for humanitarian aid, supporting healthcare efforts in disaster-stricken or underserved regions. The devices have the potential to disrupt traditional markets for blood collection and wound management devices. Their eco-friendly design and innovative features offer significant advantages over existing products.

Individuals managing diabetes through regular blood glucose monitoring can use the blotter for finger prick blood collection, making the process easier and more environmentally friendly. Patients with chronic conditions that require frequent blood testing can use the blotter to simplify home-based blood collection and reduce waste. In one embodiment, the blotter may be made available in a sterile aluminum pouch, and the same pouch may be used to send the used blotter for testing. The gentle materials and ease of use make the blotter suitable for pediatric care, where minimizing discomfort and ensuring safety are paramount. For elderly patients, the devices provide an efficient means to manage blood collection and bleeding, addressing the needs of this demographic effectively.

The following detailed description of embodiments of the invention make reference to the accompanying drawings in which like references indicate similar elements, showing by way of illustration specific embodiments of practicing the invention. Description of these embodiments is in sufficient detail to enable those skilled in the art to practice the invention. One skilled in the art understands that other embodiments may be utilized, and that logical, mechanical, electrical, functional, and other changes may be made without departing from the scope of the present invention. The following detailed description is, therefore, not to be taken in a limiting sense, and the scope of the present invention is defined only by the appended claims.

### Blotter

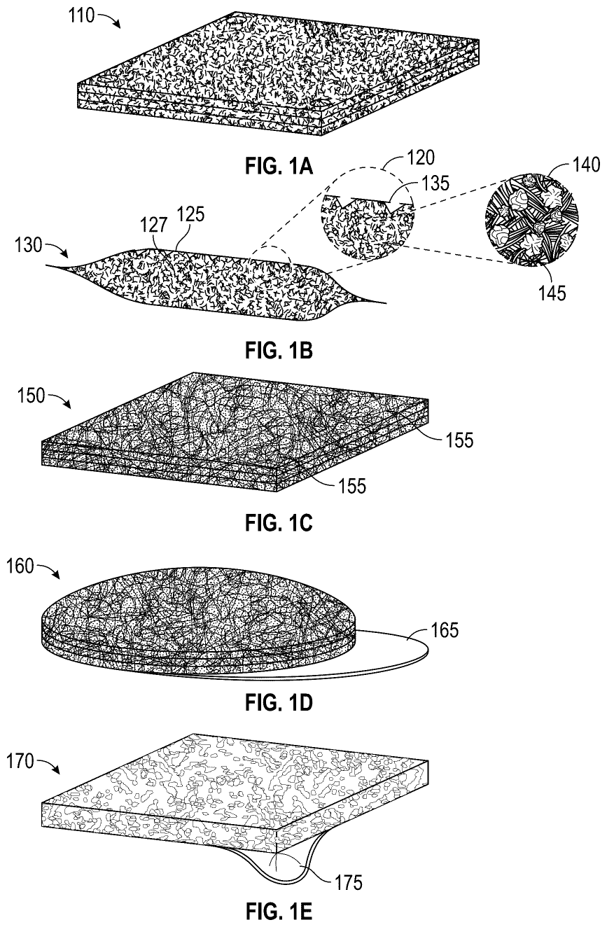

FIGS. 1A-1E illustrate embodiments of the blotter.

FIG. 1A illustrates one embodiment of the blotter including multiple sheets of cellulose material which are bonded together. The blotter is designed to be a compact, easy-to-use device for collecting blood from a finger prick or other small wound made for blood collection. Its structure consists of one or more layers of absorbent material, each contributing to the overall functionality of the device. The blotter is made of a super absorbent material such as cellulose or fluff pulp, which can rapidly absorb and retain a specific volume of blood. In one embodiment, the size of the blotter defines how much blood it can absorb. The choice of material ensures high absorbency and retention capacity, making it suitable for diagnostic purposes.

In one embodiment, the blotter 110 consists of multiple layers of absorbent material. In one embodiment, the layers of blotter 110 are laminated together using environmentally friendly adhesives that do not compromise the biodegradability of the final product. The adhesives may be starch-based, protein-based, natural rubber latex, plant-based polyurethane, or cellulose based. In one embodiment, such an adhesive may be applied in liquid or paste form to bond the outer layer and the absorbent core. The adhesive is then cured through drying, heating, or chemical reaction, resulting in a robust bond. The adhesive is selected to not interfere with the buffer or the tests to be run on the blood.

An anticoagulant layer is integrated within the absorbent core of the blotter, in one embodiment. This layer is infused with anticoagulants such as Trisodium Citrate and/or EDTA (Ethylenediaminetetraacetic acid) to prevent the collected blood from coagulating within the blotter. In one embodiment, the anticoagulants are infused directly into the absorbent material during the manufacturing process. This ensures even distribution throughout the device, providing consistent anticoagulant action. In one embodiment, the anticoagulant layer is designed to release the anticoagulant gradually as blood is absorbed, ensuring that the collected blood remains in a liquid state that is suitable for diagnostic testing.

FIG. 1B illustrates one embodiment of the blotter with an outer layer surrounding the absorbent material. The outer layer 125 of the blotter 130 is made from a permeable, biodegradable material, such as cellulose fibers, in one embodiment. This layer allows blood to pass through easily while providing a comfortable surface for the user. The core 127 of the blotter 130 in one embodiment comprises super absorbent material such as fluff pulp, which can rapidly absorb and retain a specific volume of blood.

In one embodiment, as shown in the cut-out 120, the outer layer 125 of the blotter 130 includes one-way valves 135 to allow blood to pass through. FIG. 1B also illustrates a second-level cut-out 140, zooming in to illustrate a microscopic view of cellulose fibers with embedded clumps of an anticoagulant 145, such as (tri-)sodium citrate. The image shows the intricate network of fibers with distinct granular clumps of sodium citrate scattered throughout.

FIG. 1C illustrates one embodiment of the blotter including multiple sheets with partial adhesion. In this embodiment, the sheets of the blotter 150 are fastened together at one or more individual locations 155, rather than having the entire blotter laminated. In one embodiment, the fastening may be an adhesive, a staple, a compression based fastening, a staple-less staple, a paper clinch, or any other mechanism to fasten together the sheets that make up the blotter.

FIG. 1D illustrates one embodiment of a round blotter with a tab. The blotter 160 may be fastened using an adhesive or another mechanism. In one embodiment, the blotter 160 may have a tab 165 extending from a portion of the blotter. The tab 165 provides a gripping area which does not require the user to be in physical contact with the blotter. This avoids having the user touch a potentially blood soaked blotter.

FIG. 1E illustrates one embodiment of a square blotter with a tab. The blotter 170 is a single layer in this exemplary embodiment. The blotter 170 may be fastened using an adhesive or another mechanism. In one embodiment, the blotter 170 includes a tab 175. The tab may be on a corner of the blotter 170, as shown, or positioned in another location. The tab 175 may extend along multiple sides of the blotter 170.

The tabs, illustrated in FIGS. 1D and 1E, may be used with any of the other configurations of a blotter or a clotter. In one embodiment, the tab is an extended part of a bottom layer of the blotter. In one embodiment, the bottom layer of the blotter is waxed, coated by a waterproof material, or otherwise made impervious to water/blood. In one embodiment, the bottom layer is colored. This alters the perception of color of the blotter by the user and makes the blotter more palatable to users who may not tolerate the sight of blood. The shape of the blotter is shown as round or square, however it may be any shape. furthermore, FIGS. 1A-1E illustrate various types of fibers that may be used. Any of the designs may be used with any of the fiber structures shown.

In some embodiments, the blotter size and configuration may be selected based on the tests that will be run on the blood. In one embodiment, in addition to the round/oval and square/rectangular shapes, the blotter may have any other shape. In one embodiment, the blotter shape may be based on the tests.

In one embodiment, the blotter is infused with one or more anticoagulants such as Trisodium Citrate and/or EDTA (Ethylenediaminetetraacetic acid). One or more of the following anticoagulants may be used alone or in combination with Trisodium Citrate and/or EDTA and/or each other: Heparin, Warfarin, Dabigatran, Rivaroxaban, Apixaban, Edoxaban, Fondaparinux, Enoxaparin, Dalteparin, Tinzaparin, Argatroban, Dipyridamole, Bivalirudin, Desirudin, acetylsalicylic acid, Clopidogrel, Ticagrelor, Dipyridamole, and Argatroban.

These substances prevent blood from coagulating within the blotter, ensuring that the collected blood remains usable for diagnostic testing. The integration of these anticoagulants does not interfere with the natural coagulation process at the wound site, allowing the wound to heal properly. In one embodiment, the blotter may be infused with the anticoagulant by soaking the formed blotter in anticoagulant dissolved in water, and then drying the blotter.

In one embodiment, a clotter may be included in a blood sampling kit with the blotter.

### Clotter

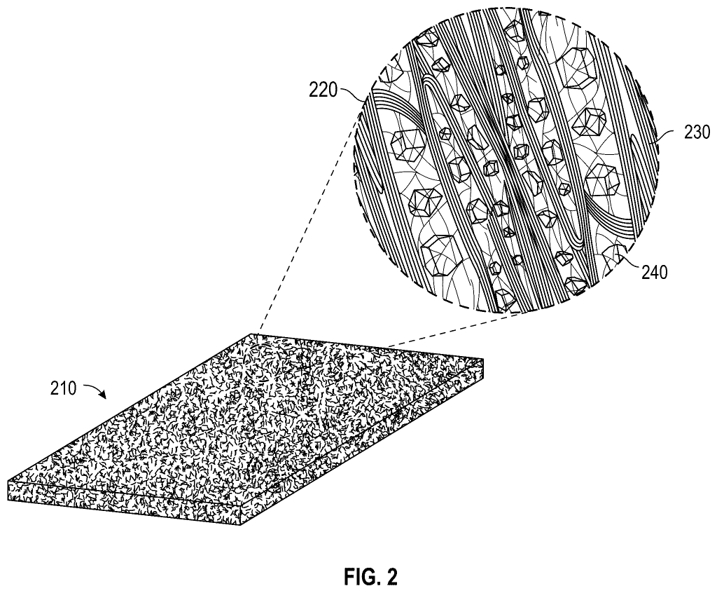

FIG. 2 illustrates one embodiment of the clotter. The clotter 210 is one or more sheets of material, such as cellulose or fluff pulp with embedded coagulants used to speed up clot formation and stop bleeding. The zoom-in 220 illustrates the material fibers 230 and the embedded coagulants 240. The integrated coagulant is designed to rapidly induce blood clotting. This makes it suitable for use in situations where quick coagulation is needed, such as in surgical settings, for hemophiliacs, or by first responders. The coagulant ensures that the wound stops bleeding quickly while protecting the area from external debris. The coagulant may be silica, collagen, cellulose, or other materials.

In one embodiment, the clotter has an outer layer made from a permeable, biodegradable material that allows for easy application and comfortable contact with the wound. The core of the clotter is composed of super absorbent materials that can quickly soak up blood while facilitating rapid coagulation, in one embodiment. An integrated coagulant layer ensures that the coagulant unit can induce clotting quickly, making it ideal for emergency use. The coagulant is infused into the absorbent material during the manufacturing process, ensuring even distribution and immediate availability upon contact with blood, in one embodiment. The coagulant layer is designed to release the coagulant rapidly upon application, promoting quick clotting and stopping the bleeding efficiently.

In one embodiment, the clotter 210 may be used for blood collection in situations where the collected blood should be clotted. This is particularly useful for serum tests, where the absence of anticoagulants and the presence of a stable clot are necessary to separate the serum from the blood cells effectively. Silica-activated tubes are used in the prior art for blood collection where clotting is desired and is designed to provide consistent and reliable clotting times, making them ideal for a wide range of clinical chemistry tests, including measurements of glucose, cholesterol, and various enzymes. This same chemical used within the large surface area of the cellulose fibers rather than in a traditional blood collection tube is thus superior in performance.

The silica particles used in a blood collector tube are typically used to later extract serum from the sample by centrifuging the sample such that serum and the clotted blood components are separated, and then the serum is used for laboratory analysis. Such silica particles may be embedded in the clotter and used for blood collection for such tests.

In one embodiment, silica or glass particles serve as clot activators. These particles are finely divided silicon dioxide, similar to the composition of sand or quartz. When added to the clotter, provide a surface that triggers the activation of clotting factors in the blood. This process, known as contact activation or the intrinsic pathway, involves several steps:
Surface Activation: The silica particles act as a surface for the adsorption of plasma proteins, including Factor XII (Hageman factor), which is a key initiator of the intrinsic coagulation pathway.
Activation Cascade: Once Factor XII binds to the silica particles, it becomes activated to Factor Xlla. This activated factor then catalyzes the activation of Factor XI to Factor Xla, continuing the cascade of reactions.
Formation of Clot: The cascade progresses through the activation of several other factors, ultimately leading to the conversion of prothrombin to thrombin. Thrombin then converts fibrinogen to fibrin, forming a stable blood clot.

The presence of silica particles accelerates this entire process, ensuring rapid clot formation.

In the case of the blotter, samples collected from a patient for the purposes of serology (diagnostic testing of the serum component of the blood), the sample do not need to be coagulated and separated, because the whole blood itself is preserved for later testing, and the mechanism of testing serological components of the blood becomes similar to that of testing fresh blood collected from a patient, where the LFA contains a Red Blood Cell (RBC) filter rather than separating them by clotting. This saves time and reduces potential human errors, by eliminating the centrifugation steps required.

The rapid clotting facilitated by silica particles helps to minimize the time required for sample processing, thereby improving the efficiency of laboratory workflows.

Although only a single configuration of the clotter is shown, one of skill in the art would understand that the shape, thickness, and size of the clotter may vary based on the intended use.

### Usage

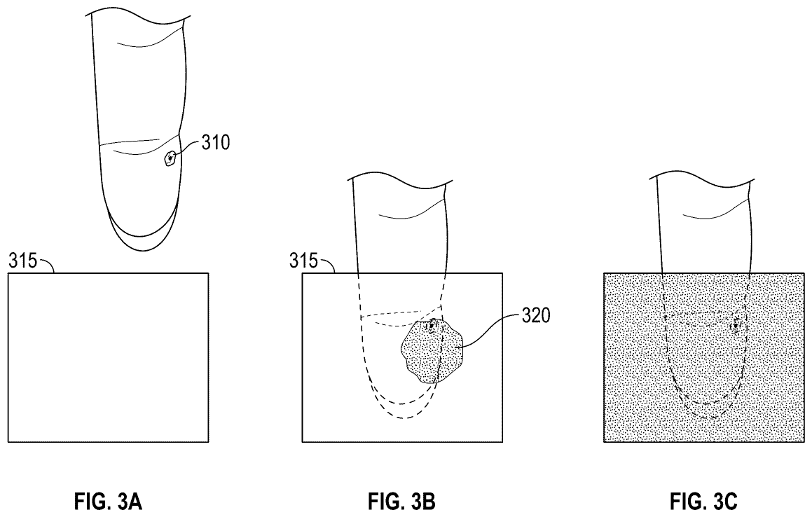

FIGS. 3A-3C illustrate a sequence of images demonstrating one embodiment of a visual indicator mechanism of the blotter, showing the color change as the device absorbs blood. The sequence of images shows one embodiment of the visual indicator mechanism of the blotter, showing the color change as the device absorbs blood.

The blotter includes a visual indicator mechanism to signal when it has absorbed a sufficient amount of blood, in one embodiment. The initial illustration in FIG. 3A shows the finger with the small wound 310-made in one embodiment with a lancet-and the blotter 315. The second illustration in FIG. 3B shows some blood 320 on the blotter, with a portion of the blotter having changed color. The final illustration in FIG. 3C shows the blotter 315 saturated with blood.

The saturation of the blotter 315 provides a visual indication mechanism to be a clear signal when the blotter has absorbed enough blood for testing, enhancing user convenience and ensuring proper blood collection.

In one embodiment, the blotter 315 is made from white material that gradually turns red as it absorbs blood. This visual change helps users determine when the blotter is saturated and ready for removal, ensuring that the appropriate amount of blood has been collected for testing. In one embodiment, the size of the blotter is selected based on the amount of blood to be collected for the specific test(s).

In one embodiment, the blotter 315 is treated with chemicals that cause the blotter material to turn yellow or orange upon contact with blood, rather than red. This feature is designed to reduce the potential for the color of blood to be off-putting or nauseating to some users. In one embodiment, TMB (3,3′,5,5′-Tetramethylbenzidine) and/or Guaiacol may be used in non-HRP-based diagnostic assays. In one embodiment, Chromotropic Acid and/or Diazonium Salts may be used. In one embodiment, Bromocresol Green may be used in non-pH-sensitive assays. In one embodiment, the color-changing chemical may be made in an aqueous solution, and the blotter may be soaked in the solution. The blotter is then dried completely. In one embodiment, the color-changing chemical may be mixed with the anticoagulant, to streamline the production process.

These alternative embodiments provide various methods to treat the blotter, offering color change upon contact with blood while considering the potential impact on diagnostic assays and user safety.

In one embodiment, a colored translucent layer on the back side of the blotter alters the color of the blood on the blotter, from the perspective of a user viewing the blotter.

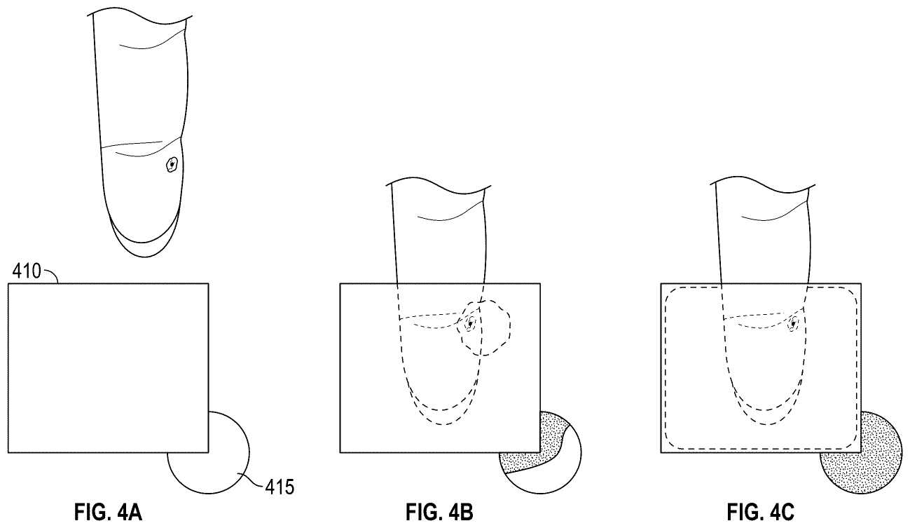

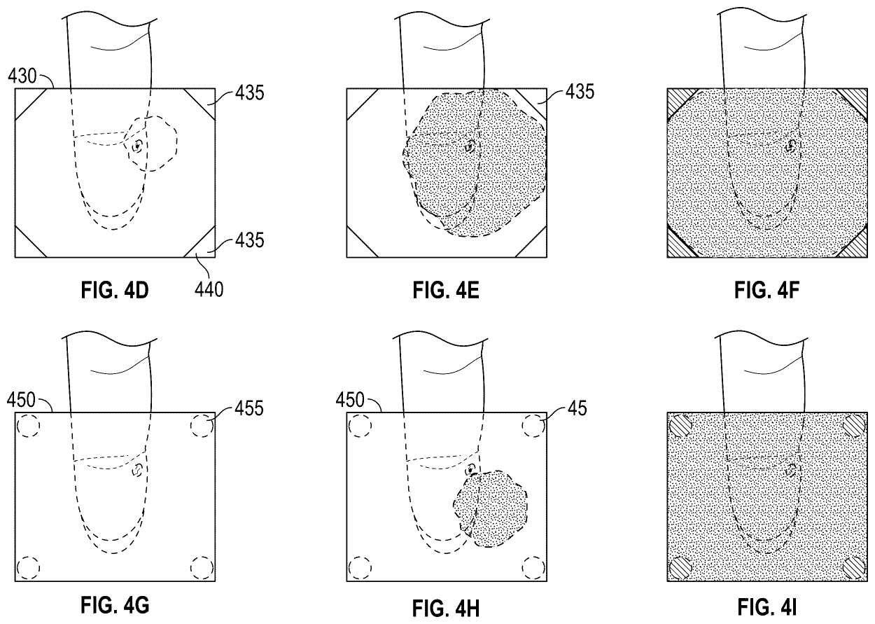

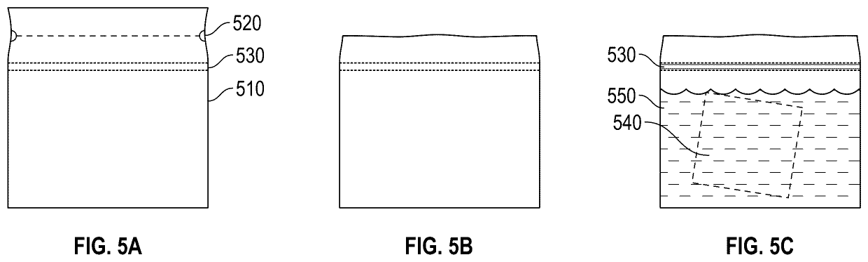

FIGS. 4A-4C illustrate a sequence of images demonstrating another embodiment of the visual indicator mechanism of the blotter. In this embodiment, a tab 415 or other component extending from the blotter 410 is used to provide the physical indication. The first illustration shows the blotter 410 and tab 415, before it is used, then as the blotter starts filling, and finally when the blotter is saturated. When the entire tab 415 is the same color, it indicates that the blotter has absorbed enough blood.

FIGS. 4D-4F illustrate a sequence of images demonstrating another embodiment of the visual indicator mechanism of the blotter. In this embodiment, the blotter 430 has visual indicators 435 in one or more corners 440. The visual indicators 435 change color as the blotter is saturated.

FIGS. 4G-4I illustrate a sequence of images demonstrating another embodiment of the visual indicator mechanism of the blotter. In this embodiment, the blotter 450 has visual indicators 455 in one or more locations within the blotter. In one embodiment, the indicators are in one or more corners of the blotter 450. However, the indicators may be located at any position on the blotter 450. For example, there may be a central indicator. The visual indicator 455 changes color as the blotter is saturated.

### Resealable and Recyclable Pouch

To further reduce environmental impact, in one embodiment, the system is packaged in environmentally friendly materials.

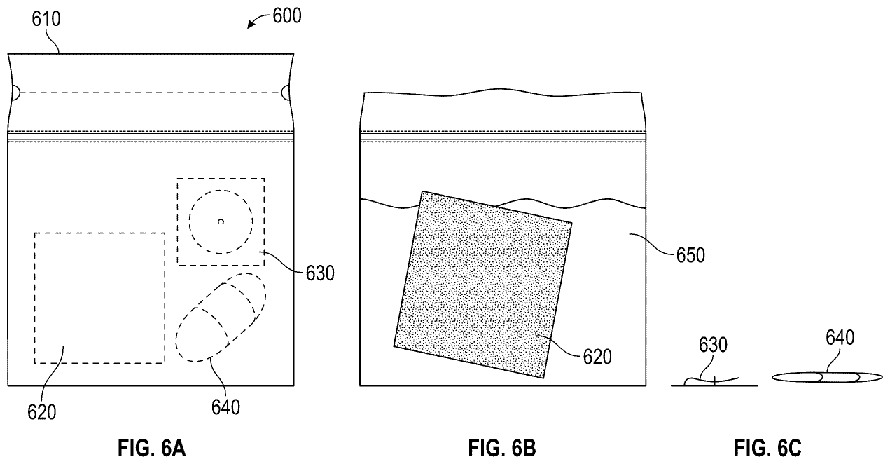

FIGS. 5A-5C illustrate a sequence of images demonstrating use of a transport pouch, including a tear-off section and resealing strip.

In one embodiment, the clotter or blotter is delivered in a sterile pouch 510 that ensures product sterility while being recyclable. In one embodiment, the pouch 510 is made of aluminum. This packaging choice aligns with the overall goal of minimizing environmental footprint and promoting sustainability. The sterile pouch 510 used for packaging the blood collection device is manufactured through a process that ensures sterility.

In one embodiment, the aluminum pouch 510 is made from rolled aluminum sheets that are subjected to high temperatures during manufacturing, inherently sterilizing the material. The pouch is then formed and sealed using heat, which further ensures that the pouch remains sterile. This process eliminates the need for additional sterilization steps, as the high temperatures involved in the rolling, forming, and heat-sealing processes effectively maintain the sterility of the pouch until it is used.

In one embodiment, the aluminum pouch 510 includes a top which is sealed at the factory. In one embodiment, there is a perforation 520, which enables easy tearing off of a tear-off section of the aluminum pouch 510. In one embodiment, the pouch 510 also includes a resealing strip 530. In one embodiment, the resealing strip 530 is an interlocking plastic groove and ridge (commonly referred to as ZIPLOCK™) is used to create a resealable pouch 510. In another embodiment it may be an adhesive or other type of resealing strip which can be used to securely reseal the aluminum pouch 510. When the user receives the sealed pouch 510 shown in FIG. 5A, they tear off the top portion, using perforation 520. They can then open the pouch 510 and remove the blotter and/or clotter. FIG. 5B illustrates the aluminum pouch 510 after the top has been removed.

After use, the blotter 540 can be placed into an aluminum pouch 510 that contains a buffer solution 550 to preserve the blood sample during transportation. The pouch 510 features a tear-off section indicating it has not been used before, and a resealing strip 530 for secure closure. In one embodiment, the aluminum pouch 510 used to return the blotter may be a different pouch than the pouch in which the blotter is stored prior to use.

In one embodiment, if the blotter is designed to be returned to a facility for testing, the aluminum pouch may also be used to securely mail or otherwise transfer the collected blood in the blotter to an appropriate location. As shown in FIG. 5C, the blotter 540 can be placed in the pouch 510 and sealed using the resealing strip 530. In one embodiment, the blotter 540 may be placed in a buffer solution 550. In one embodiment, the aluminum pouch 510 may be pre-printed with the address of the facility to which the package should be returned.

The pouches are designed to maintain the sterility of the blotter until use. They protect the blotter from contaminants and ensure that it is safe for use in medical settings.

The pouches can be recycled after use to minimize medical waste, in one embodiment. Although the sterile pouch 510 is described as an aluminum pouch, in one embodiment, it may be made of a different material. For example, the material may be a metal alloy with aluminum, plastic, biodegradable plant based materials, wax coated cardboard or paper, or another material.

### Blood Sampling Kit

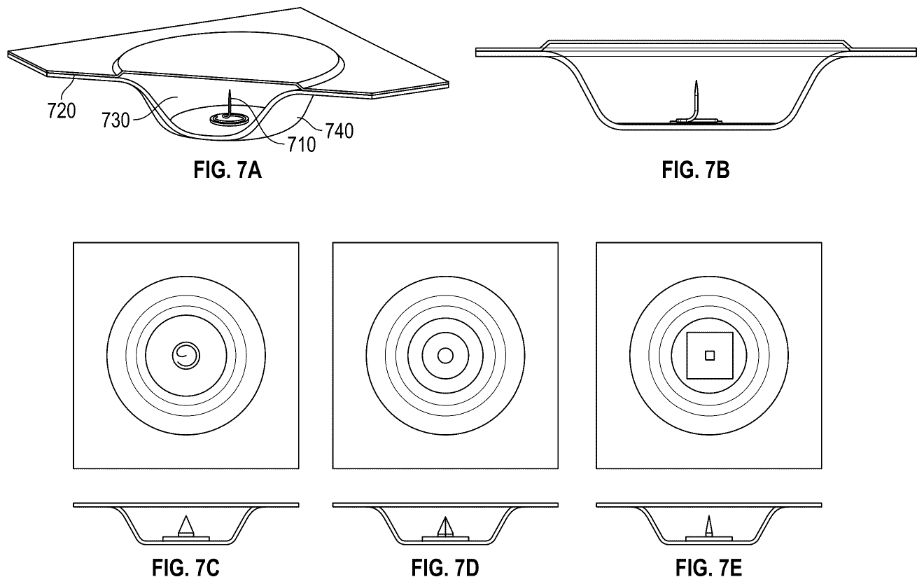

FIGS. 6A-6C are illustrations of a blood sampling kit, prior to use and after use.

FIG. 6A illustrates the blood sampling kit 600 includes in one embodiment a sealed aluminum pouch 610, which includes the blotter 620, a lancet 630, and a buffer solution capsule or ampule 640 with a buffer solution. FIG. 6B illustrates the sealed aluminum pouch 610, with the used blotter 620, floating in the buffer solution 650.

The buffer solution 650 is to preserve the blood sample during transportation. In one embodiment, the buffer solution 650 contains lysing agents and antimicrobials to ensure that the blood sample remains preserved and non-infectious during transportation. This allows patients to send their blood samples to a central lab without leaving their homes, reducing healthcare costs and improving convenience. The capsule may be added into the pouch. In one embodiment, once the pouch is sealed, the user squeezes the pouch to burst or otherwise open the capsule inside. The preservation buffer solution 650 is released from the capsule, filling the pouch and ensuring the blood sample is preserved without the risk of spilling.

The blood sampling kit 600 may optionally include a clotter (not shown).

The blotter 620 and/or clotter are removed. Optionally the lancet 630 is removed. Optionally, the capsule 630 with the buffer solution is removed. The lancet 630 then can be used to make a small wound (finger prick) so the blotter 620 can be used to collect blood for testing. The lancet 630 may be attached to the pouch, or a separate unit which is removed from the pouch upon opening. The blotter can then be placed within the pouch, and the pouch released. In one embodiment, the blotter 620 is within a separate envelope prior to use, to ensure sterility. This separate envelope may be a smaller aluminum pouch with a perforation for opening, but without the resealing capability. FIG. 6C illustrates the optionally discarded portions of the blood collection kit, the used lancet 630 and the emptied capsule 640. In one embodiment, the capsule 640 is a gel capsule. In one embodiment, the capsule is popped. The capsule may be popped in the pouch, after the pouch is sealed, in one embodiment. In another embodiment, the capsule is popped or otherwise prior to the sealing, and the buffer solution 650 is emptied into the pouch 610.

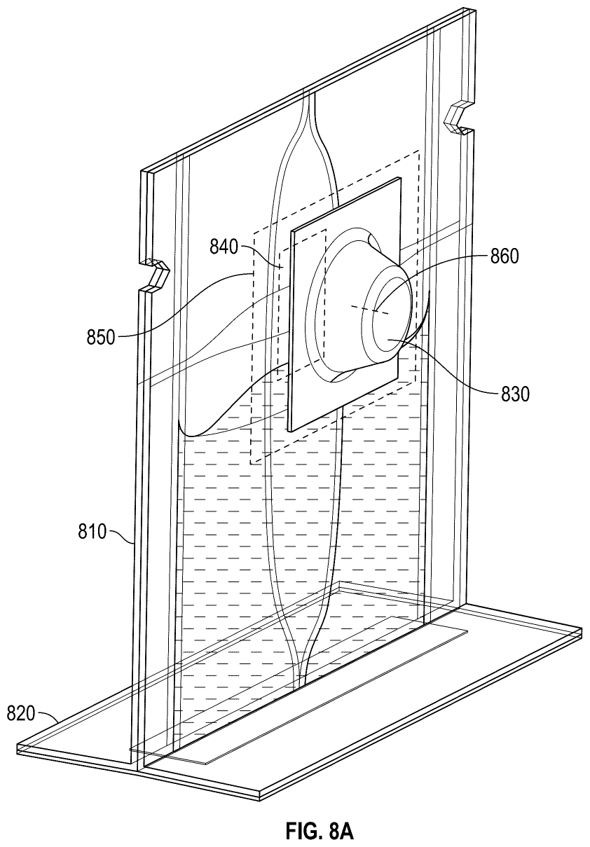

In one embodiment, the lancet enclosed in the sampling kit is inside a protective bubble 740 as shown in FIGS. 7A-7E. The lancet includes a sharp component 710 enclosed in an aluminum foil package 720, in one embodiment. FIGS. 7A-7E depict five different embodiments of the lancet that show the use of a stainless steel needle, a ceramic element needle, and an aluminum formed needle in both top views, and side section views. In one embodiment, the sharp component 710 has a groove, so the sharp component 710 does not need to be removed to collect the blood from the finger prick.

In one embodiment, the cavity 730 is filled with a liquid, such as ethanol or formaldehyde for keeping the inside sterilized and purged with nitrogen to avoid chemical reactions (such as oxidation) of the needle. In another embodiment, the cavity 730 may be filled with a gas or other material.

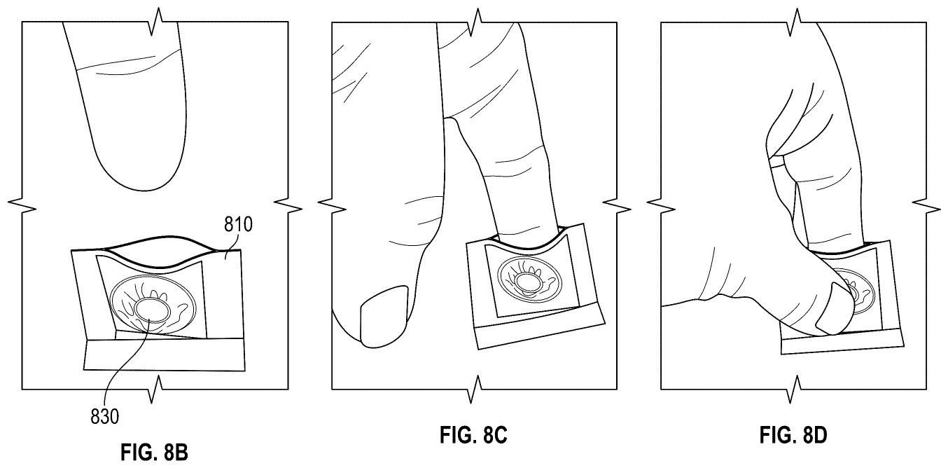

In one embodiment, the lancet 710 is integrated with the aluminum resealable bag or pouch. FIG. 8A illustrates one embodiment of the use of the aluminum resealable bag with integral lancet.

In one embodiment, the aluminum resealable pouch 810 includes a foot portion 820 that allows the bag 810 to be stood up. The blister 830 including the lancet 860 is attached to the outside of one side of the pouch 810. The lancet 860 can be used to pierce the skin, so blood can be collected on a blotter 850, within the bag. This enables blood collection without interacting with the bloody blotter.

In one embodiment, on the far side of the bag 810, corresponding to the location of the lancet, there is a reinforced portion 840 of the aluminum pouch 810. This ensures that the lancet 860 does not pierce through the pouch. The blister 830 in one embodiment is attached via an adhesive to the pouch 810. In another embodiment, the blister 830 is formed integrally with the pouch 810. In one embodiment, in use the lancet 860 pierces through the first side of the pouch 810, but the reinforced portion 840 ensures that it does not pierce through the other side. Because the lancet does not pierce through the other side of the pouch, and the lancet attachment is secure and watertight, neither side of the pouch is damaged by the use of the lancet. This means that the pouch remains still watertight, allowing the buffer to be used. In one embodiment, the pouch 810 is an aluminum pouch, with an aluminum blister 830. In another embodiment, the pouch 810 may be made of other materials. In one embodiment, the pouch may not include a foot portion 820.

FIGS. 8B-8D illustrate one embodiment of the sequence of using the pouch with an integral lancet. After the aluminum packaging is opened as shown in FIG. 8B, a finger is inserted, as shown in FIG. 8C, and then with a second finger the blister 830 containing the lancet is pressed to engage the needle, as shown in FIG. 8D. The needle penetrates a layer of the aluminum bag to which it is attached, and enters the user's finger, and causes the finger to produce drops of blood internal to the aluminum packaging and drip blood onto the blotter within the aluminum bag. In this embodiment, the user does not remove the blotter from the pouch. Instead, the user inserts their finger into the opened pouch, allowing the blotter to absorb blood while still inside the pouch. This method maintains sterility and reduces the risk of contamination.

### Manufacturing Process

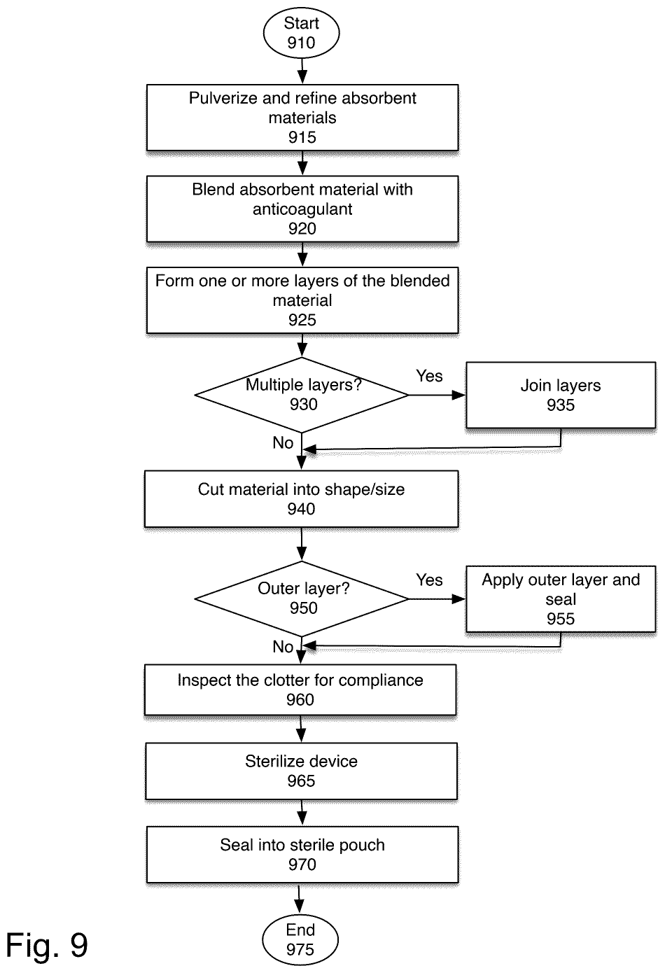

FIG. 9 is a flowchart of one embodiment of manufacturing the blood sampling kit. The process illustrated is for manufacturing and assembling a blood sampling kit including a blotter. One of skill in the art would understand that the process would work similarly for manufacturing the clotter. The production process for the blotter involves several steps designed to ensure the quality and functionality of the clotter. The process starts at block 910.

In preparation for manufacturing, in one embodiment, at block 915 cellulose fibers and fluff pulp are pulverized and refined to achieve the desired consistency and absorbency. In one embodiment, this process is similar to the way various types and grades of paper are made. Alternatively, ready-made paper products can be used as the base material. In one embodiment, the paper type products that are made for absorbing blood may be used as the base material for the production.

At block 920, absorbent materials are blended with the appropriate amounts of anticoagulants ensuring even distribution throughout the core of the device. In one embodiment, the appropriate amounts of anticoagulants are diluted in a liquid, such as sterile deionized water, and sheets of the absorbent material is then placed into the liquid, or the liquid is sprayed onto the absorbent material. The material is then dried in a drying process causing the water in the liquid to substantially evaporate, leaving only the anticoagulant in its dried form distributed throughout the fibers within the material. In one embodiment, a color changing chemical is blended with the anticoagulants, to also embed that into the device. Alternatively, the material before being cut or after cutting may be sprayed or imprinted with the color changing material.

At block 925, in one embodiment, one or more layers are formed from the blended absorbent and anticoagulant material and layered according to design specifications. The design specification in one embodiment includes the thickness of the layer(s). In one embodiment, the design includes at least one layer. In one embodiment, the design includes multiple layers.

If the design includes multiple layers, as determined at block 930, the layers are joined at block 935. In one embodiment, the layers are laminated together using environmentally friendly adhesives that do not compromise the biodegradability of the final product. The adhesives may be starch-based, protein-based, natural rubber latex, plant-based polyurethane, or cellulose based. The starch-based adhesives are composed primarily of amylose and amylopectin, which provide strong adhesion properties. The adhesive is then cured through drying or heating, resulting in a robust and biodegradable bond. Protein-based adhesives are derived from natural proteins such as casein (from milk) and soy protein. These adhesives contain polypeptides and amino acids that provide strong adhesion properties. Natural rubber latex adhesives are derived from the sap of rubber trees (Hevea brasiliensis). These adhesives consist of polyisoprene, which provides excellent elasticity and adhesion properties. Plant-based polyurethane adhesives are synthesized from bio-based polyols derived from vegetable oils such as castor oil, soybean oil, and palm oil. These adhesives provide strong bonding properties and are designed to be more environmentally friendly than traditional petroleum-based polyurethanes. Cellulose-based adhesives are derived from natural cellulose fibers obtained from plants. These adhesives consist of cellulose derivatives such as carboxymethyl cellulose (CMC) and hydroxyethyl cellulose (HEC), which provide excellent adhesion properties. Other types of adhesives may be used.

In one embodiment, such an adhesive may be applied in liquid or paste form to bond the outer layer and the absorbent core. The adhesive is then cured through drying, heating, or chemical reaction, resulting in a robust bond. The adhesive is selected to not interfere with the buffer or the tests to be run on the blood. In another embodiment, staples or other attachment elements may be used. In another embodiment, paper clinching techniques may be used, which use the sheets to self-attach in a staple-less connection. In another embodiment, an outer layer is used to surround the absorbent materials, and the adhesive is used only on the outer layer.

At block 940, the resulting material is cut into the specific shapes and sizes required for the particular test(s). In one embodiment, the size of the devices may depend on their use. The sizes for blotter may range from 5 mm by 5 mm to 50 mm by 50 mm, depending on the amount of blood to be collected. In one embodiment, the blotter is designed to collect 1to 50 microliters of blood. For the clotter, for example, an in-home first aid kit may include Band-Aid™ sized clotters, while an EMT's kit may include larger clotters, up to full body size.

In some embodiments, the layers may be joined after the cutting.

If there is an outer layer, as determined at block 950, the outer layer is applied at block 955. In one embodiment, the outer layer is a closely woven cellulose material. In one embodiment, the outer layer includes perforations. In one embodiment, the outer layer includes one-way valve perforations, so that blood can enter the absorbent material, but will not exit through the perforations. In one embodiment, the outer layer surrounds the blended absorbent and anticoagulant material.

At block 960, each piece is inspected for consistency and adherence to design specifications.

At block 965, the device is sterilized. Sterilization is a critical step to ensure the safety and efficacy of the devices. In one embodiment, each device undergoes gamma irradiation, a proven method for sterilizing medical devices without compromising the integrity of the materials. In another embodiment, Ethylene Oxide (EtO) Sterilization may be used, for materials that may be sensitive to radiation. This process ensures that all components are free from microbial contamination.

At block 970, after sterilization, the devices are immediately sealed in sterile pouches to maintain sterility until use. In one embodiment, the sterile pouches are designed with a double-sealing mechanism, including a first tear-off section and a resealing strip, to ensure that the devices remain sterile during transportation and storage, and the same pouch can be used to contain a used device. The process then ends at block 975.

In one embodiment, to ensure the highest standards of quality and safety, the following quality control measures are implemented:
Raw materials are inspected upon arrival to ensure they meet quality standards before entering the production process.
Each step of the production process is monitored, with regular inspections to detect and correct any deviations from the specifications.
Finished devices undergo absorbency tests to ensure they meet the required performance standards.
The efficacy of the integrated anticoagulants and coagulants is tested to ensure proper functionality.
Final products are tested for sterility to confirm that they are free from microbial contamination.

In one embodiment, detailed batch records are maintained for every production run, ensuring traceability and accountability for each device produced. In one embodiment, the production facility operates under strict quality management systems, adhering to ISO 13485 and other relevant medical device standards.

In one embodiment, the devices are subjected to a series of absorbency tests to verify their performance. For example, using a controlled environment, the devices are exposed to simulated blood to measure their absorption capacity. The volume of blood absorbed and retained by each device is recorded and compared to the design specifications. In one embodiment, the devices are also tested with real human blood samples to ensure that their performance in clinical settings matches the results obtained with simulated blood, in one embodiment.

### Conclusion

Thus, the present system provides a blood collection system which includes a blotter in a sterile pouch, the blotter impregnated with anticoagulants, to maintain collected blood in a liquid state, to enable testing for various conditions. The system may further have the sterile pouch as a reusable pouch, which can be used to return the blotter to a laboratory for testing. In one embodiment, a buffer may be used in the pouch with the used blotter, to stabilize the blood. The blotter may provide visual feedback, to indicate when sufficient blood is collected. This visual feedback may be the color of the blotter, or a portion of the blotter or a tab integral with the blotter. In one embodiment, the blood collection system may further include a lancet, to create a small wound for collecting the blood. In one embodiment, the blood collection system may additionally or alternatively include a clotter, to provide fast clotting of the blood. This may be useful for persons with bleeding disease, or for other wounds.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative rather than a restrictive sense.

1. A blood collection system comprising a blood collection device comprising:
an outer layer made of a permeable, biodegradable material;
an absorbent core composed of a super absorbent material;
an anticoagulant integrated within the absorbent core;
the blood collection device to collect blood for testing and maintain the blood in a liquid form after collection.

2. The blood collection system of claim 1, wherein the anticoagulant is selected from the group consisting of Trisodium Citrate and Ethylenediaminetetraacetic acid (EDTA).

3. The blood collection system of claim 1, further comprising;
a visual indicator mechanism that changes color as the blood collection device absorbs blood.

4. The blood collection system of claim 1, wherein the super absorbent material is selected from cellulose fibers and fluff pulp.

5. The blood collection system of claim 1, wherein the outer layer and absorbent core are laminated together using an environmentally friendly adhesive.

6. The blood collection system of claim 1, further comprising:
a sterile aluminum pouch for packaging the blood collection device, wherein the pouch is recyclable and maintains the sterility of the device until use.

7. The blood collection system of claim 6, further comprising:
a buffer solution with lysing agents and antimicrobials to preserve the blood in the blood collection device during transportation, the buffer solution released into the sterile aluminum pouch prior to sealing the sterile aluminum pouch.

8. The blood collection system of claim 7, further comprising:
a capsule containing the buffer solution, wherein the buffer solution is opened to release the buffer solution into the sterile aluminum pouch.

9. A blotter comprising:
super absorbent material;
an anticoagulant integrated into the super absorbent material;
a visual indicator mechanism that changes color as the blotter absorbs blood, to indicate when sufficient blood has been absorbed;
the blotter to collect blood for testing and maintain the blood in a liquid form after collection.

10. The blotter of claim 9, wherein the visual indicator mechanism comprises the blotter changing color.

11. The blotter of claim 9, further comprising:
an outer layer made of permeable material.

12. The blotter of claim 11, further comprising:
wherein the outer layer and absorbent core are laminated together using an environmentally friendly adhesive.

13. The blotter of claim 9, wherein the super absorbent material comprises a plurality of sheets of cellulose material.

14. The blotter of claim 13, wherein the plurality of sheets is joined together using one or more of: lamination, adhesive, staples, and staple-less connection.

15. A blood testing kit comprising:
a blotter comprising:
super absorbent material;
an anticoagulant integrated into the super absorbent material;
an aluminum pouch configured to maintain the blotter in a sterile condition prior to use, the aluminum pouch further configured to receive the blotter after the blotter is used to collect blood sample for testing;
a buffer solution to preserve the blood sample in the blotter during transportation.

16. The blood testing kit of claim 15, further comprising:
a clotter comprising:
a super absorbent material;
a coagulant integrated into the super absorbent material;
such that the clotter stops bleeding on contact by speeding up clot formation.

17. The blood testing kit of claim 15, wherein the blotter further comprises an outer layer made of a permeable, biodegradable material.

18. The blood testing kit of claim 17, wherein the outer layer and absorbent core are laminated together using an environmentally friendly adhesive.

19. The blood testing kit of claim 15, further comprising:
a capsule containing the buffer solution, the capsule configured to be opened to release the buffer solution to preserve the blood sample for testing.

20. The blood testing kit of claim 15, further comprising:
a lancet integrated with the aluminum pouch.
